(19) United States
(12) Patent Application Publication
(10) Pub. No.: US 2023/0003678 A1
(43) Pub. Date: Jan. 5, 2023

(54) POTENTIAL MEASUREMENT DEVICE

(71) Applicant: SONY SEMICONDUCTOR SOLUTIONS CORPORATION, KANAGAWA (JP)

(72) Inventor: YOSHIHISA MATOBA, KANAGAWA (JP)

(21) Appl. No.: 17/757,220

(22) PCT Filed: Nov. 17, 2020
(86) PCT No.: PCT/JP2020/042790

§ 371 Date: Jun. 10, 2022

(30) Foreign Application Priority Data
Dec. 19, 2019 (JP) 2019-229453

(51) Int. Cl. G01N 27/30; G01N 27/416

(52) U.S. Cl. CPC G01N 27/30; G01N 27/4161

(57) ABSTRACT

To provide a potential measurement device capable of keeping a temperature of a cell and/or a culture solution (in particular, a temperature of a cell) constant. Provided is a potential measurement device including a semiconductor substrate; a wiring layer on the semiconductor substrate; a first electrode on the wiring layer; and a second electrode configured to detect an action potential of a cell on the wiring layer. A temperature measurement unit is formed on the semiconductor substrate. A heat conduction unit and a plurality of wirings connected to the second electrode are formed in the wiring layer.

### TECHNICAL FIELD

The present technology relates to a potential measurement device.

### BACKGROUND ART

There are potential measurement devices in which minute reading electrodes are arranged in an array form to electrochemically measure potentials generated due to chemical changes of solutions on the reading electrodes. For example, a potential measurement device in which biological cells with which a culture solution are filled are put on reading electrodes and an action potential generated by the biological cells is measured was proposed (for example, see PTL 1).

In particular, a potential measurement device in which electrodes, amplifiers, and A/D converters, and the like are integrated in one semiconductor substrate (chip) using a complementary metal oxide semiconductor (CMOS) integrated circuit technology and potentials are measured simultaneously at multiple points has attracted attention.

### CITATION LIST

### Patent Literature

[PTL 1]
JP 2002-31617 A

### SUMMARY

### Technical Problem

However, in a situation in which there are restrictions on a temperature of a cell on an electrode and/or a culture solution because of an organism, there is concern of the temperature of cell on the electrode and/or a culture solution (in particular, a temperature of a cell) not being kept constant in the technology proposed in PTL 1.

Accordingly, the present technology has been devised in view of such circumstances, and a main objective of the present technology is to provide a potential measurement device capable of keeping a temperature of a cell and/or a culture solution (in particular, a temperature of a cell) constant.

### Solution to Problem

The present inventors and others have successfully kept a temperature of a cell and/or a culture solution (in particular, a temperature of a cell) constant as an outcome of an earnest study carried out to achieve the above-described objective and have finalized the present technology.

That is, according to a first aspect of the present technology,

a potential measurement device includes a semiconductor substrate, a wiring layer on the semiconductor substrate, a first electrode on the wiring layer, and a second electrode configured to detect an action potential of a cell on the wiring layer.
A temperature measurement unit is formed on the semiconductor substrate.
A heat conduction unit and a plurality of wirings connected to the second electrode are formed in the wiring layer.

In the potential measurement device according to the first aspect of the present technology, the heat conduction unit and the first electrode may be connected.

In the potential measurement device according to the first aspect of the present technology, and

the heat conduction unit may be formed to extend to a region near the temperature measurement unit.

In the potential measurement device according to the first aspect of the present technology,

the heat conduction unit and the first electrode may be connected, and

The heat conduction unit is formed to extend from the first electrode to a region near the temperature measurement unit.

In the potential measurement device according to the first aspect of the present technology,

the heat conduction unit may include first and second heat conduction members, and
the first and second heat conduction members may be connected.

In the potential measurement device according to the first aspect of the present technology,

the heat conduction unit may include first and second heat conduction members,
the first electrode and the first heat conduction member may be connected, and
the first and second heat conduction members may be connected.

In the potential measurement device according to the first aspect of the present technology,

the plurality of wirings may each be connected through a via,
the heat conduction unit may include first and second heat conduction members,
the first and second heat conduction members may be connected,
the first heat conduction member and the via may be formed in substantially the same layer, and
the second heat conduction member and the wiring may be formed in substantially the same layer.

In the potential measurement device according to the first aspect of the present technology,

the plurality of wirings may each be connected through a via,
the heat conduction unit may include first and second heat conduction members,
the first and second heat conduction members may be connected,
the first heat conduction member and the via may be formed in substantially the same layer,
the second heat conduction member and the wiring may be formed in substantially the same layer,
the first electrode and the first heat conduction member may be connected, and
the first and second heat conduction members may be connected.

In the potential measurement device according to the first aspect of the present technology,

the heat conduction unit may have a groove extending downward and a heat conduction material in which the groove is buried.

In the potential measurement device according to the first aspect of the present technology,

the heat conduction unit may have a groove extending downward and a heat conduction material in which the groove is buried, and
the heat conduction material and the first electrode may be connected.

The potential measurement device according to the first aspect of the present technology may further include an electrode region,

in the electrode region, a plurality of the second electrodes may be arranged in a 2-dimensional array form, and
at least one first electrode may be arranged on an outer circumference of the electrode region.

The potential measurement device according to the first aspect of the present technology may further include an electrode region,

in the electrode region, a plurality of the second electrodes may be arranged in a 2-dimensional array form, and
at least one first electrode may be arranged on an outer circumference of the electrode region, and
the heat conduction unit and said at least one first electrode may be connected.

The potential measurement device according to the first aspect of the present technology may further include an electrode region,

in the electrode region, at least one said first electrode and a plurality of the second electrodes may be arranged in a 2-dimensional array form.

The potential measurement device according to the first aspect of the present technology may further include an electrode region,

in the electrode region, at least one said first electrode and a plurality of the second electrodes may be arranged in a 2-dimensional array form, and
the heat conduction unit and said at least one first electrode may be connected.

In the potential measurement device according to the first aspect of the present technology,

at least two second electrode may be included, and
at least one first electrode may be arranged between said at least two electrodes.

In the potential measurement device according to the first aspect of the present technology,

at least two second electrode may be included, and
at least one first electrode may be arranged between said at least two electrodes, and
the heat conduction unit and said at least one first electrode may be connected.

According to a second aspect of the present technology,

A potential measurement device including;

a semiconductor substrate;
a wiring layer on the semiconductor substrate; and
a third electrode configured to detect an action potential of a cell on the wiring layer,
wherein a temperature measurement unit is formed on the semiconductor substrate, and
wherein a plurality of wirings connected to a heat conduction unit and the third electrode are formed in the wiring layer.

In the potential measurement device according to the second aspect of the present technology, the heat conduction unit and the third electrode may be connected.

In the potential measurement device according to the second aspect of the present technology,

the heat conduction unit may be formed to extend to a region near the temperature measurement unit.

In the potential measurement device according to the second aspect of the present technology,

the heat conduction unit and the third electrode may be connected, and
the heat conduction unit may be formed to extend from the third electrode to a region near the temperature measurement unit.

According to the present technology, it is possible to keep a temperature of a cell and/or a culture solution (in particular, a temperature of a cell) constant. The advantageous effects mentioned here are not necessarily limited and may be any advantageous effects obtained in the present disclosure.

### BRIEF DESCRIPTION OF DRAWINGS

FIG. 1 is a sectional view illustrating an exemplary configuration of a potential measurement device according to a first embodiment to which the present technology is applied.

FIG. 2 is a sectional view illustrating an exemplary configuration of a potential measurement device according to a second embodiment to which the present technology is applied.

FIG. 3 is a sectional view illustrating an exemplary configuration of a potential measurement device according to a third embodiment to which the present technology is applied.

FIG. 4 is a sectional view illustrating an exemplary configuration of a potential measurement device according to a fourth embodiment to which the present technology is applied.

FIG. 5 is a plan view illustrating an arrangement example of a first electrode (temperature measurement dummy electrode) and a second electrode (reading electrode) included in a potential measurement device according to a fifth embodiment to which the present technology is applied.

FIG. 6 is a plan view illustrating an arrangement example of a first electrode (temperature measurement dummy electrode) and a second electrode (reading electrode) included in a potential measurement device according to a sixth embodiment to which the present technology is applied.

FIG. 7 is a plan view illustrating an arrangement example of a first electrode (temperature measurement dummy electrode) and a second electrode (reading electrode) included in a potential measurement device according to a seventh embodiment to which the present technology is applied.

FIG. 8 is a diagram illustrating an exemplary configuration of a potential measurement device according to the present technology.

### DESCRIPTION OF EMBODIMENTS

Hereinafter, preferred modes for carrying out the present technology will be described. The embodiments to be described below are exemplary representative examples of the present technology, and thus the scope of the present technology is not to be construed narrowly thereby. In the description of the drawings, unless otherwise mentioned, “up” means an up direction in the drawing, “down” means a down direction, “left” means the left direction of the drawing, and “right” means a right direction in the drawing.

Description will be made in the following order.
1. Overview of present technology
2. First embodiment (Example 1 of potential measurement device)
3. Second embodiment (Example 2 of potential measurement device)
4. Third embodiment (Example 3 of potential measurement device)
5. Fourth embodiment (Example 4 of potential measurement device)
6. Fifth embodiment (Example 5 of potential measurement device)
7. Sixth embodiment (Example 6 of potential measurement device)
8. Seventh embodiment (Example 7 of potential measurement device)

### 1. Overview of Present Technology

First, an overview of the present technology will be described.

There are device in which minute electrodes (reading electrodes) are arrayed side by side and potentials of solutions on the electrodes are electrochemically measured. Of the devices, there is a device (a potential measurement device) in which biological cells with which a culture solution is filled and which are put on minute electrodes and an action potential generated by the biological cells is measured.

In particular, a potential measurement device in which electrodes, amplifiers, and AD converters, and the like are integrated in one chip using a CMOS integrated circuit technology and potentials are measured simultaneously at multiple points has attracted attention.

When an action potential of cells is measured using the device (the potential measurement device), it is necessary to cultivate cells on electrodes on the front surface of a device chip and maintain an action thereof. On the other hand, to perform temperature measurement of a chip and control in a CMOS integrated circuit, a thermometer in which intersection points formed on a semiconductor (silicon (Si)) substrate are used is used.

As described above, to cultivate cells and maintain an action thereof on a device chip (for example, on a wiring layer of a potential measurement device) measuring an action potential of the cells, it is necessary to keep a temperature of a cell (a culture solution) at a constant temperature (for example, 37±0.5° C.). On the other hand, on the device side, a chip temperature increases due to power consumption during an operation. Accordingly, by monitoring a chip temperature with a thermometer inside the chip and feeding back to an external cooling mechanism (for example, a Peltier element or the like), it is possible to perform control of the chip temperature. In this case, however, since a thermometer formed on a semiconductor substrate (a silicon substrate) and a chip surface (a chip surface facing cells) on which the cells are put are located at distant positions, an error may occur between a surrounding temperature of the cells and a temperature measured by the thermometer in some cases.

An insulation film (an inter-layer film: for example, SiO, SiN, or the like) between a thermometer and a surface electrode has thermal resistance or thermal capacitance to some extent. Therefore, when temperature control is performed through feedback, it may be difficult to keep a temperature of the chip surface on which cells are put within a specified range in some cases due to occurrence of delay of an increase or decrease in temperature, overshooting, or undershooting.

The present technology has been devised in view of the foregoing circumstances. According to the present technology, a potential measurement device includes a semiconductor substrate, a wiring layer on the semiconductor substrate, a first electrode (a temperature measurement dummy electrode) on the wiring layer, and a second electrode (a reading electrode) configured to detect an action potential of a cell on the wiring layer. A temperature measurement unit is formed on the semiconductor substrate. A plurality of wirings connected to a heat conduction unit and the second electrode are formed in the wiring layer. For example, the heat conduction unit and the first electrode may be connected and may be formed to extend from the first electrode to a region near the temperature measurement unit.

According to the present technology, by forming the heat conduction unit, heat can be transported to the vicinity of the temperature measurement unit formed in the semiconductor substrate (a silicon substrate) (for example, an interface between the semiconductor substrate and the wiring layer). Thus, it is possible to measure a temperature of a chip surface on which a cell is put with high accuracy. Accordingly, in surface temperature control feedback of an increase in a chip temperature during an operation of a chip (the potential measurement device) or a change in an environmental temperature, it is possible to perform control of a small change with high accuracy and achieve stabilization of a cell action.

Any material may be selected freely as long as a material of which the heat conduction unit is formed is a material that has low thermal resistance and/or high thermal conductivity. For example, a metal material may be used. A material of which the heat conduction unit is formed may be a material that has low thermal resistance and/or high thermal conductivity and has high electrical resistance (has insulation).

Since the first electrode (the temperature measurement dummy electrode) and the second electrode (the reading electrode) are formed in substantially the same layer on the wiring layer (on the potential measurement device or the chip), it is preferable to form of the same material from the viewpoint of manufacturing applicability. However, the present technology is not limited thereto. A material of which the first electrode (the temperature measurement dummy electrode) is formed may differ from a material of which the second electrode (the reading electrode) is formed.

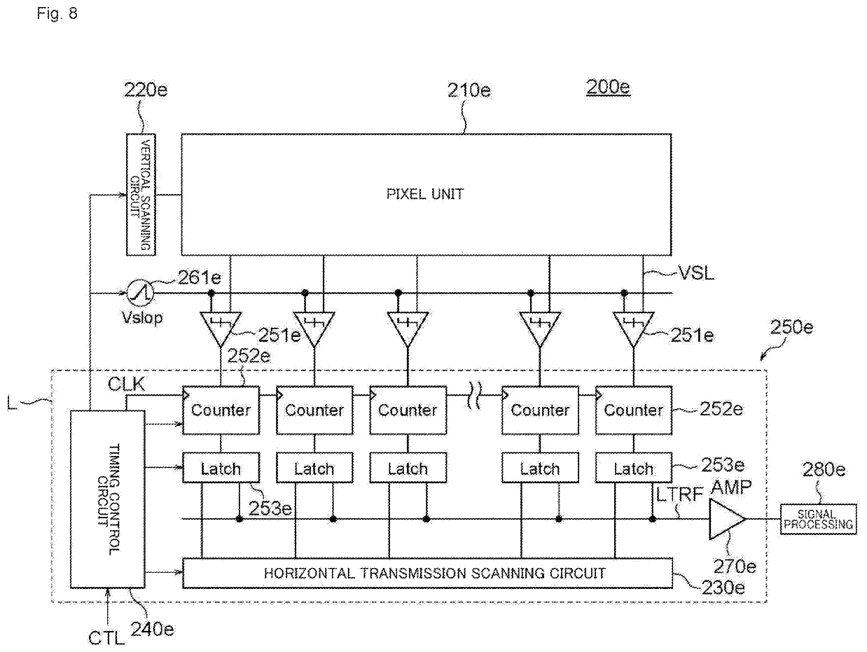

Next, an overall exemplary configuration of the potential measurement device according to the present technology will be described with reference to FIG. 8.

FIG. 8 is a diagram illustrating an exemplary configuration of a potential measurement device 200e according to the present technology. An L region illustrated in FIG. 8 is a region in which a logic chip is configured.

The potential measurement device 200e illustrated in FIG. 8 includes a cell array unit (a pixel unit) 210e, a vertical scanning circuit 220e, a horizontal transmission scanning circuit 230e, a timing control circuit 240e, and an ADC group 250e serving as a pixel signal reading unit.

The potential measurement device 200e includes a DAC including a digital-analog conversion device (DAC) 261e, a bias circuit, an amplifier circuit (S/A) 270e, and a signal processing circuit 280e. Of these constituent elements, the cell array unit 210e, the vertical scanning circuit 220e, the horizontal transmission scanning circuit 230e, the ADC group 250e, the DAC, the bias circuit, and the amplifier circuit (S/A) 270e are configured with analog circuits. The timing control circuit 240e and the signal processing circuit 280 are configured with digital circuits.

The cell array unit (the pixel unit) 210e has an electrode region where a plurality of second electrodes (reading electrodes) detecting action potentials of cells or a plurality of first electrodes (temperature measurement dummy electrodes) and a plurality of second electrodes (reading electrodes) detecting action potentials of cells are arranged in a 2-dimensional array form, a region where a differential amplifier circuit including an amplifier transistor that multiplies a potential difference between a reading electrode and a reference electrode and outputs the amplified potential difference is formed, a region in which a heat conduction unit is formed, a region in which a temperature measurement unit is formed, and the like.

The timing control circuit 240e generates an internal clock as a control circuit that sequentially reads signals of the cell array unit (the pixel unit) 210e. The vertical scanning circuit 220e controls a row address or row scanning of the cell array unit (the pixel unit) 210. The horizontal transmission scanning circuit 230e controls a column address or column scanning of the cell array unit (the pixel unit) 210e.

The ADC group 250e is formed from a plurality of A/D conversion circuits. Each A/D conversion circuit includes a comparator 251e that compares a reference voltage Vslop which has a ramp waveform (RAMP) in which a reference voltage generated by the DAC 261e is changed stepwise with an analog signal (a potential VSL) passing through a vertical signal line from pixels of each row line. Further, each A/D conversion circuit includes a counter 252e that counts a comparison time and a latch 253e that retains a count result.

The ADC group 250e has an n-bit digital signal conversion function and is arranged at each vertical signal line (a column line), and a column-parallel ADC block is configured. An output of each latch 253e is connected to, for example, a horizontal transmission line LTRF with a 2n-bit width. In addition, 2n amplifier circuits 270e corresponding to the horizontal transmission line LTRF and a signal processing circuit 280e are arranged.

Hereinafter, preferred modes for carrying out the present technology will be described in detail with reference to the drawings. Embodiments to be described below are exemplary representative examples of the present technology, and thus the scope of the present technology is not construed narrowly.

### 2. First Embodiment (Example 1 of Potential Measurement Device)

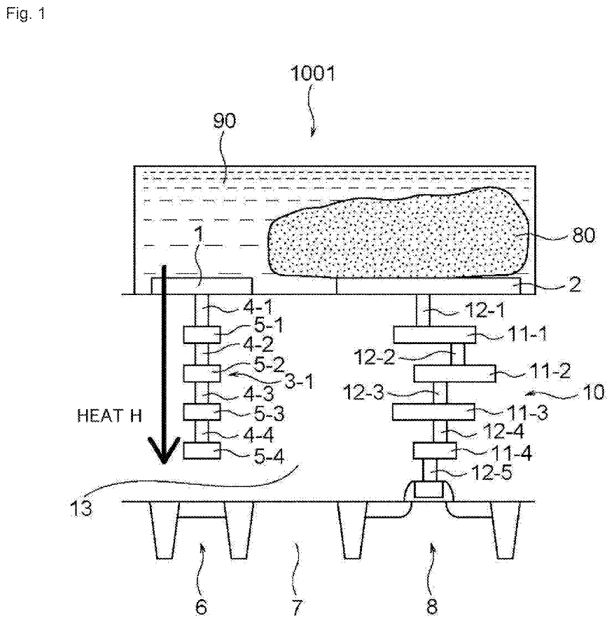

A potential measurement device according to a first embodiment (Example 1 of a potential measurement device) according to the present technology will be described with reference to FIG. 1.

FIG. 1 is a sectional view illustrating an exemplary configuration of a potential measurement device according to the first embodiment of the present technology and is specifically a sectional view of a potential measurement device 1001.

The potential measurement device 1001 includes a semiconductor substrate 7, a wiring layer 10 on the semiconductor substrate 7, a first electrode (a temperature measurement dummy electrode) 1 on the wiring layer 10, and a second electrode (a reading electrode) 2 on the wiring layer 10. A temperature measurement unit 6 is formed on the semiconductor substrate 7, and a heat conduction unit 3-1 is formed to be buried in an insulation film 13 (an inter-layer film: for example, SiO, SiN, or the like) and a plurality of wirings 11-1 to 11-4 connected to the second electrode 2 are formed in the wiring layer 10. The second electrode (the reading electrode) 2 can detect an action potential of a cell 80 on the second electrode 2 cultivated in a culture solution 90.

The heat conduction unit 3-1 includes four first heat conduction members 4-1 to 4-4 and four second heat conduction members 5-1 to 5-4 in FIG. 1. As illustrated in FIG. 1, the first electrode (a temperature measurement dummy electrode) 1 and the first heat conduction member 4-1 are connected, the first heat conduction member 4-1 and the second heat conduction member 5-1 are connected, the second heat conduction member 5-1 and the first heat conduction member 4-2 are connected, the first heat conduction member 4-2 and the second heat conduction member 5-2 are connected, the second heat conduction member 5-2 and the first heat conduction member 4-3 are connected, the first heat conduction member 4-3 and the second heat conduction member 5-3 are connected, the second heat conduction member 5-3 and the first heat conduction member 4-4 are connected, and the first heat conduction member 4-4 and the second heat conduction member 5-4 are connected. That is, a starting end of the heat conduction unit 3-1 is the first heat conduction member 4-1 connected to the first electrode (a temperature measurement dummy electrode) 1, an ending end of the heat conduction unit 3-1 is the second heat conduction member 5-4, and the heat conduction unit 3-1 is formed to extend in a down direction (a down direction in FIG. 1) from the first electrode (a temperature measurement dummy electrode) 1 to a region near the temperature measurement unit 6.

As illustrated in FIG. 1, the second electrode (the reading electrode) 2 and the plurality of wirings 11-1 to 11-4 are connected, and the amplifier (the amplifier transistor) 8 and the plurality of wirings 11-1 to 11-4 are connected.

Specifically, the second electrode (the reading electrode) 2 and a via 12-1 are connected, the via 12-1 and the wiring 11-1 are connected, the wiring 11-1 and a via 12-2 are connected, the via 12-2 and the wiring 11-2 are connected, the wiring 11-2 and a via 12-3 are connected, the via 12-3 and the wiring 11-3 are connected, the wiring 11-3 and a via 12-4 are connected, the via 12-4 and the wiring 11-4 are connected, the wiring 11-4 and a via 12-5 are connected, and the via 12-5 and the amplifier (the amplifier transistor) 8 are connected.

In the potential measurement device 1001, the heat conduction member 3-1 is formed so that heat advances in the direction indicated by an arrow (a heat conduction path H) and transport to the vicinity of the temperature measurement unit 6. Accordingly, a temperature of a surface and/or the vicinity of the surface of the chip (the wiring layer 10) corresponding to a range in which the cell 80 (the culture solution 90) is located can be measured with high accuracy. An interval (a distance in the right and left directions in FIG. 1) between the first electrode (a temperature measurement dummy electrode) 1 and the second electrode (the reading electrode) 2 may be any interval. However, to measure a temperature of a surface (a right surface in FIG. 1) and/or the vicinity of the surface (the right surface in FIG. 1) of the chip (the wiring layer 10) corresponding to the range in which the cell 80 (the culture solution 90) is located with higher accuracy, the interval (the distance in the right and left directions in FIG. 1) between the first electrode (the temperature measurement dummy electrode) 1 and the second electrode (the reading electrode) 2 is preferably shorter.

The content described above in the potential measurement device according to the first embodiment (Example 1 of the potential measurement device) of the present technology can be applied to potential measurement devices according to the second to seventh embodiments of the present technology to be described as far as there is particularly no technical contradiction.

### 3. Second Embodiment (Example 2 of Potential Measurement Device)

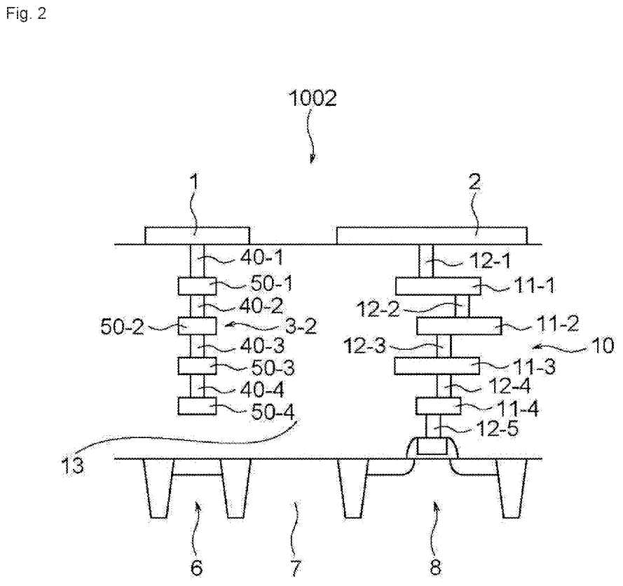

A potential measurement device according to a second embodiment (Example 2 of a potential measurement device) according to the present technology will be described with reference to FIG. 2.

FIG. 2 is a sectional view illustrating an exemplary configuration of a potential measurement device according to the second embodiment of the present technology and is specifically a sectional view of a potential measurement device 1002.

The potential measurement device 1002 includes the semiconductor substrate 7, the wiring layer 10 on the semiconductor substrate 7, the first electrode (a temperature measurement dummy electrode) 1 on the wiring layer 10, and the second electrode (a reading electrode) 2 capable of detecting an action potential of a cell on the wiring layer 10. A temperature measurement unit 6 is formed on the semiconductor substrate 7, and a heat conduction unit 3-2 is formed to be buried in an insulation film 13 (an inter-layer film: for example, SiO, SiN, or the like) and a plurality of wirings 11-1 to 11-4 connected to the second electrode 2 are formed in the wiring layer 10.

The heat conduction unit 3-2 includes four first heat conduction members 40-1 to 40-4 and four second heat conduction members 50-1 to 50-4 in FIG. 2. As illustrated in FIG. 2, the first electrode (a temperature measurement dummy electrode) 1 and the first heat conduction member 40-1 are connected, the first heat conduction member 40-1 and the second heat conduction member 50-1 are connected, the second heat conduction member 50-1 and the first heat conduction member 40-2 are connected, the first heat conduction member 40-2 and the second heat conduction member 50-2 are connected, the second heat conduction member 50-2 and the first heat conduction member 40-3 are connected, the first heat conduction member 40-3 and the second heat conduction member 50-3 are connected, the second heat conduction member 50-3 and the first heat conduction member 40-4 are connected, and the first heat conduction member 40-4 and the second heat conduction member 50-4 are connected. That is, a starting end of the heat conduction unit 3-2 is the first heat conduction member 40-1 connected to the first electrode (a temperature measurement dummy electrode) 1, an ending end of the heat conduction unit 3-2 is the second heat conduction member 50-4, and the heat conduction unit 3-2 is formed to extend in a down direction (a down direction in FIG. 2) from the first electrode (a temperature measurement dummy electrode) 1 to a region near the temperature measurement unit 6.

As illustrated in FIG. 2, the second electrode (the reading electrode) 2 and the plurality of wirings 11-1 to 11-4 are connected, and the amplifier (the amplifier transistor) 8 and the plurality of wirings 11-1 to 11-4 are connected.

Specifically, the second electrode (the reading electrode) 2 and a via 12-1 are connected, the via 12-1 and the wiring 11-1 are connected, the wiring 11-1 and a via 12-2 are connected, the via 12-2 and the wiring 11-2 are connected, the wiring 11-2 and a via 12-3 are connected, the via 12-3 and the wiring 11-3 are connected, the wiring 11-3 and a via 12-4 are connected, the via 12-4 and the wiring 11-4 are connected, the wiring 11-4 and a via 12-5 are connected, and the via 12-5 and the amplifier (the amplifier transistor) 8 are connected.

In FIG. 2, the first heat conduction member 40-1 and the via 12-1 are formed in substantially the same layer, the second heat conduction member 50-1 and the wiring 11-1 are formed in substantially the same layer, the first heat conduction member 40-2 and the via 12-2 are formed in substantially the same layer, the second heat conduction member 50-2 and the wiring 11-2 are formed in substantially the same layer, the first heat conduction member 40-3 and the via 12-3 are formed in substantially the same layer, the second heat conduction member 50-3 and the wiring 11-3 are formed in substantially the same layer, the first heat conduction member 40-4 and the via 12-4 are formed in substantially the same layer, and the second heat conduction member 50-4 and the wiring 11-4 are formed in substantially the same layer. From the viewpoint of manufacturing applicability, the first heat conduction member and the via formed in substantially the same layer and/or the second heat conduction member and the wiring formed in substantially the same layer are preferably formed of the same material (for example, a metal material), but the present technology is not limited thereto. The first heat conduction member and the via formed in substantially the same layer and/or the second heat conduction member and the wiring formed in substantially the same layer are preferably formed of different materials.

In the potential measurement device 1002, the heat conduction unit 3-2 is formed so that heat advances in the down direction (the down direction in FIG. 2) and transport to the vicinity of the temperature measurement unit 6. Accordingly, a temperature of a surface and/or the vicinity of the surface of the chip (the wiring layer 10) corresponding to a range in which a cell (the culture solution) is located can be measured with high accuracy. An interval (a distance in the right and left directions in FIG. 2) between the first electrode (the temperature measurement dummy electrode) 1 and the second electrode (the reading electrode) 2 may be any interval. However, to measure a temperature of a surface (a right surface in FIG. 2) and/or the vicinity of the surface (the right surface in FIG. 2) of the chip (the wiring layer 10) corresponding to the range in which the cell (the culture solution) is located with higher accuracy, the interval (the distance in the right and left directions in FIG. 2) between the first electrode (the temperature measurement dummy electrode) 1 and the second electrode (the reading electrode) 2 is preferably shorter.

The content described above in the potential measurement device according to the second embodiment (Example 2 of the potential measurement device) of the present technology can be applied to the potential measurement device according to the above-described first embodiment and potential measurement devices according to the third to seventh embodiments of the present technology to be described below as far as there is particularly no technical contradiction.

### 4. Third Embodiment (Example 3 of Potential Measurement Device)

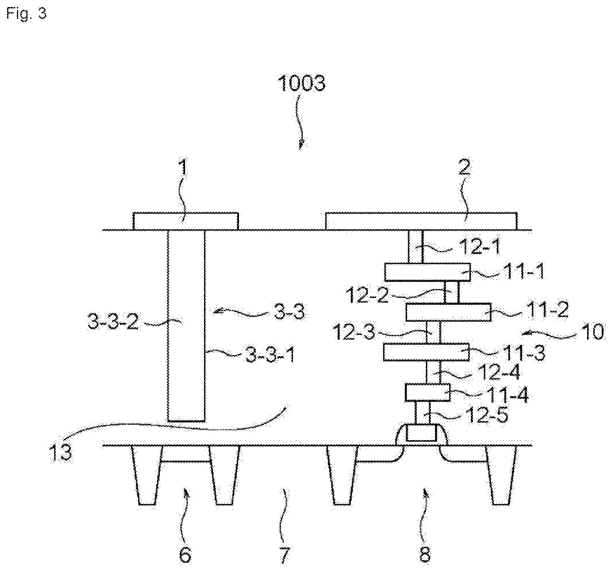

A potential measurement device according to a third embodiment (Example 3 of a potential measurement device) according to the present technology will be described with reference to FIG. 3.

FIG. 3 is a sectional view illustrating an exemplary configuration of a potential measurement device according to the third embodiment of the present technology and is specifically a sectional view of a potential measurement device 1003.

The potential measurement device 1003 includes the semiconductor substrate 7, the wiring layer 10 on the semiconductor substrate 7, the first electrode (a temperature measurement dummy electrode) 1 on the wiring layer 10, and the second electrode (a reading electrode) 2 capable of detecting an action potential of a cell on the wiring layer 10. A temperature measurement unit 6 is formed on the semiconductor substrate 7, and a heat conduction unit 3-3 is formed in the wiring layer 10, and a plurality of wirings 11-1 to 11-4 connected to the second electrode 2 are formed.

The heat conduction unit 3-3 includes a groove 3-3-1 extending downward (the down direction in FIG. 3) and a heat conduction material 3-3-2 with which the grove 3-3-1 is buried. As illustrated in FIG. 3, the first electrode (a temperature measurement dummy electrode) 1 and the heat conduction material 3-3-2 are connected. The heat conduction unit 3-3 is formed to extend from the first electrode (a temperature measurement dummy electrode) 1 to a region near the temperature measurement unit 6 in the down direction (the down direction in FIG. 3).

As illustrated in FIG. 3, the second electrode (the reading electrode) 2 and the plurality of wirings 11-1 to 11-4 are connected, and the amplifier (the amplifier transistor) 8 and the plurality of wirings 11-1 to 11-4 are connected.

Specifically, the second electrode (the reading electrode) 2 and a via 12-1 are connected, the via 12-1 and the wiring 11-1 are connected, the wiring 11-1 and a via 12-2 are connected, the via 12-2 and the wiring 11-2 are connected, the wiring 11-2 and a via 12-3 are connected, the via 12-3 and the wiring 11-3 are connected, the wiring 11-3 and a via 12-4 are connected, the via 12-4 and the wiring 11-4 are connected, the wiring 11-4 and a via 12-5 are connected, and the via 12-5 and the amplifier (the amplifier transistor) 8 are connected.

In the potential measurement device 1003, the heat conduction unit 3-3 is formed so that heat advances in the down direction (the down direction in FIG. 3) and transport to the vicinity of the temperature measurement unit 6. Accordingly, a temperature of a surface and/or the vicinity of the surface of the chip (the wiring layer 10) corresponding to a range in which a cell (the culture solution) is located can be measured with high accuracy. An interval (a distance in the right and left directions in FIG. 3) between the first electrode (the temperature measurement dummy electrode) 1 and the second electrode (the reading electrode) 2 may be any interval. However, to measure a temperature of a surface (a right surface in FIG. 3) and/or the vicinity of the surface (the right surface in FIG. 3) of the chip (the wiring layer 10) corresponding to the range in which the cell (the culture solution) is located with higher accuracy, the interval (the distance in the right and left directions in FIG. 3) between the first electrode (the temperature measurement dummy electrode) 1 and the second electrode (the reading electrode) 2 is preferably shorter.

The content described above in the potential measurement device according to the third embodiment (Example 3 of the potential measurement device) of the present technology can be applied to the potential measurement device according to the above-described first and second embodiments and potential measurement devices according to the fourth to seventh embodiments of the present technology to be described below as far as there is particularly no technical contradiction.

### 5. Fourth Embodiment (Example 4 of Potential Measurement Device)

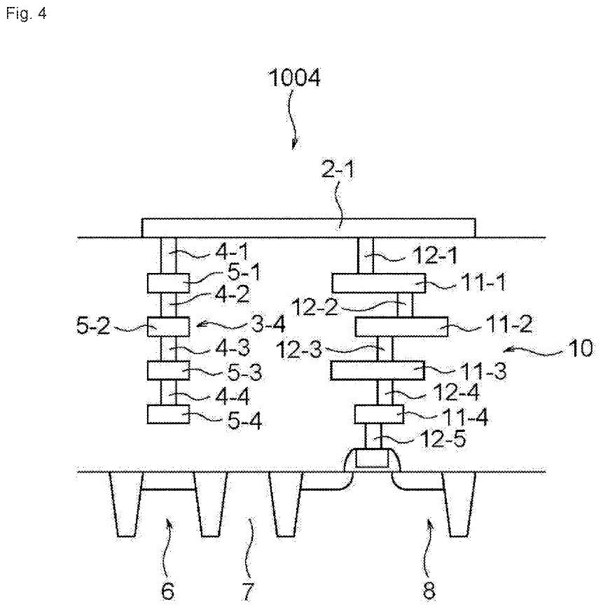

A potential measurement device according to a fourth embodiment (Example 4 of a potential measurement device) according to the present technology will be described with reference to FIG. 4.

FIG. 4 is a sectional view illustrating an exemplary configuration of a potential measurement device according to the fourth embodiment of the present technology and is specifically a sectional view of a potential measurement device 1004.

The potential measurement device 1004 includes the semiconductor substrate 7, the wiring layer 10 on the semiconductor substrate 7, the third electrode (a reading electrode) 2-1 capable of detecting an action potential of a cell on the wiring layer 10. A temperature measurement unit 6 is formed on the semiconductor substrate 7, and a heat conduction unit 3-4 is formed in the wiring layer 10, and the plurality of wirings 11-1 to 11-4 connected to the third electrode 2-1 are formed.

The heat conduction unit 3-4 includes four first heat conduction members 4-1 to 4-4 and four second heat conduction members 5-1 to 5-4 in FIG. 4. As illustrated in FIG. 4, the third electrode (a reading electrode) 2-1 and the first heat conduction member 4-1 are connected, the first heat conduction member 4-1 and the second heat conduction member 5-1 are connected, the second heat conduction member 5-1 and the first heat conduction member 4-2 are connected, the first heat conduction member 4-2 and the second heat conduction member 5-2 are connected, the second heat conduction member 5-2 and the first heat conduction member 4-3 are connected, the first heat conduction member 4-3 and the second heat conduction member 5-3 are connected, the second heat conduction member 5-3 and the first heat conduction member 4-4 are connected, and the first heat conduction member 4-4 and the second heat conduction member 5-4 are connected. That is, a starting end of the heat conduction unit 3-4 is the first heat conduction member 4-1 connected to the third electrode (the reading electrode) 2-1, an ending end of the heat conduction unit 3-4 is the second heat conduction member 5-4, and the heat conduction unit 3-4 is formed to extend in a down direction (a down direction in FIG. 4) from the third electrode (the reading electrode) 2-1 to a region near the temperature measurement unit 6. The third electrode (the reading electrode) 2-1 is connected to the heat conduction unit 3-4 and also functions as a temperature measurement dummy electrode. Instead of the heat conduction unit 3-4, the heat conduction unit 3-2 used in the potential measurement device 1002 of the second embodiment or the heat conduction unit 3-3 used in the potential measurement device 1003 of the third embodiment may be applied to the potential measurement device 1004 of the fourth embodiment.

As illustrated in FIG. 4, the third electrode (the reading electrode) 2-1 and the plurality of wirings 11-1 to 11-4 are connected, and the amplifier (the amplifier transistor) 8 and the plurality of wirings 11-1 to 11-4 are connected.

Specifically, the third electrode (the reading electrode) 2-1 and a via 12-1 are connected, the via 12-1 and the wiring 11-1 are connected, the wiring 11-1 and a via 12-2 are connected, the via 12-2 and the wiring 11-2 are connected, the wiring 11-2 and a via 12-3 are connected, the via 12-3 and the wiring 11-3 are connected, the wiring 11-3 and a via 12-4 are connected, the via 12-4 and the wiring 11-4 are connected, the wiring 11-4 and a via 12-5 are connected, and the via 12-5 and the amplifier (the amplifier transistor) 8 are connected.

In the potential measurement device 1004, the heat conduction member 3-4 is formed so that heat advances downward (the down direction in FIG. 4) and transport to the vicinity of the temperature measurement unit 6. The third electrode (the reading electrode) also functions as a temperature measurement dummy electrode and there is continuity in heat connection. Accordingly, a temperature of a surface (the right surface in FIG. 4) of a chip (the wiring layer 10) corresponding to a range in which a cell (the culture solution) is located and/or the vicinity of the surface (the right surface in FIG. 4) can be measured with high accuracy.

The content described above in the potential measurement device according to the fourth embodiment (Example 4 of the potential measurement device) of the present technology can be applied to the potential measurement device according to the above-described first to third embodiments and potential measurement devices according to the fifth to seventh embodiments of the present technology to be described below as far as there is particularly no technical contradiction.

### 6. Fifth Embodiment (Example 5 of Potential Measurement Device)

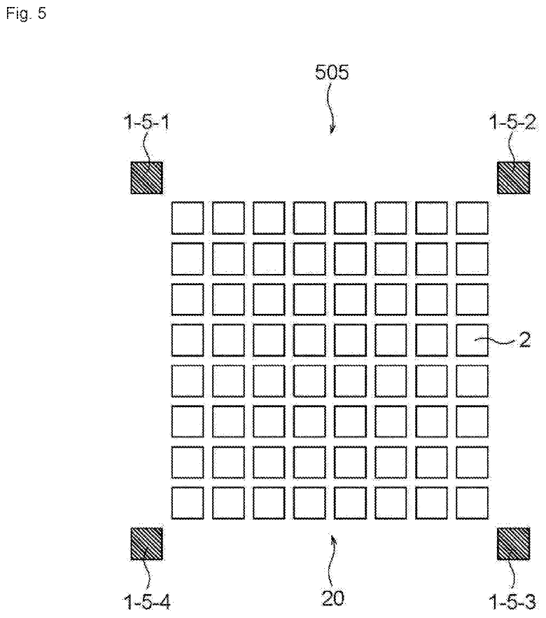

A potential measurement device according to a fifth embodiment (Example 5 of a potential measurement device) according to the present technology will be described with reference to FIG. 5.

FIG. 5 is a plan view illustrating an arrangement example of first electrodes (temperature measurement dummy electrodes) and second electrodes (reading electrodes) included in a potential measurement device according to the fifth embodiment to which the present technology is applied, and is a plan view of an arrangement configuration 505 of the first electrodes (the temperature measurement dummy electrodes) and the second electrodes (the reading electrodes) in detail.

In the arrangement configuration 505, the plurality of second electrodes (the reading electrodes) 2 are arranged in a 2-dimensional array form, and an electrode region 20 is formed.

In FIG. 5, a first electrode (a temperature measurement dummy electrode) 1-5-1 is arranged at the top left of the outer circumference of the electrode region 20, a first electrode (a temperature measurement dummy electrode) 1-5-2 is arranged at the top right of the outer circumference of the electrode region 20, a first electrode (a temperature measurement dummy electrode) 1-5-3 is arranged at the bottom right of the outer circumference of the electrode region 20, and a first electrode (a temperature measurement dummy electrode) 1-5-4 is arranged at the bottom left of the outer circumference of the electrode region 20.

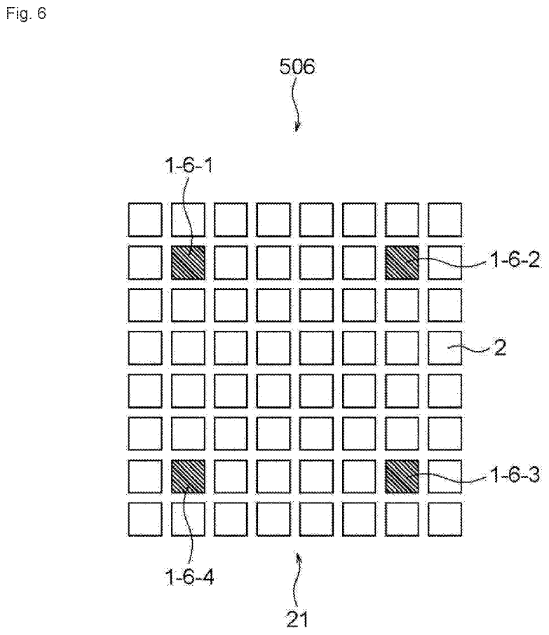

The positions of the four first electrodes (the temperature measurement dummy electrodes) 1-5-1 to 1-5-4 disposed in the electrode region 20 are not limited to the positions illustrated in FIG. 6 as long as the positions of the first electrodes are in the outer circumference of the electrode region 20. The number of first electrodes (the temperature measurement dummy electrodes) 1 is not limited to the number (four) of the first electrodes (the temperature measurement dummy electrodes) 1-5 illustrated in FIG. 6.

As the heat conduction units used in the potential measurement device according to the fifth embodiment (Example 5 of a potential measurement device) according to the present technology, the heat conduction unit 3-1 used in the potential measurement device 1001 according to the first embodiment of the present technology, the heat conduction unit 3-2 used in the potential measurement device 1002 according to the second embodiment of the present technology, or the heat conduction unit 3-3 used in the potential measurement device 1003 according to the third embodiment of the present technology may be applied.

A reference electrode used in the potential measurement device according to the fifth embodiment (Example 5 of a potential measurement device) of the present technology may be arranged in, for example, a region in the outer circumference of the electrode region 20 and a region other than the region in which the first electrodes (the temperature measurement dummy electrodes) 1-5-1 to 1-5-4 are arranged.

The content described above in the potential measurement device according to the fifth embodiment (Example 5 of the potential measurement device) of the present technology can be applied to the potential measurement device according to the above-described first to fourth embodiments and potential measurement devices according to the sixth and seventh embodiments of the present technology below to be described as far as there is particularly no technical contradiction.

### 7. Sixth Embodiment (Example 6 of Potential Measurement Device)

A potential measurement device according to a sixth embodiment (Example 6 of a potential measurement device) according to the present technology will be described with reference to FIG. 6.

FIG. 6 is a plan view illustrating an arrangement example of first electrodes (temperature measurement dummy electrodes) and second electrodes (reading electrodes) included in a potential measurement device according to the sixth embodiment to which the present technology is applied, and is a plan view of an arrangement configuration 506 of the first electrodes (the temperature measurement dummy electrodes) and the second electrodes (the reading electrodes) in detail.

In the arrangement configuration 506, four first electrodes (temperature measurement dummy electrodes) 1-6-1 to 1-6-4 and a plurality of second electrodes (the reading electrodes) 2 are arranged in a 2-dimensional array form, and an electrode region 21 is formed.

Specifically, in FIG. 6, the first electrode (the temperature measurement dummy electrode) 1-6-1 is arranged at a second row of the electrode region 21 from the top of FIG. 6 and at a second column of the electrode region 21 from the left of FIG. 6, the first electrode (the temperature measurement dummy electrode) 1-6-2 is arranged at a second row of the electrode region 21 from the top of FIG. 6 and at a second column of the electrode region 21 from the right of FIG. 6, the first electrode (the temperature measurement dummy electrode) 1-6-3 is arranged at a second row of the electrode region 21 from the bottom of FIG. 6 and at a second column of the electrode region 21 from the right of FIG. 6, and the first electrode (the temperature measurement dummy electrode) 1-6-4 is arranged at a second row of the electrode region 21 from the bottom of FIG. 6 and at a second column of the electrode region 21 from the left of FIG. 6.

The positions of the four first electrodes (the temperature measurement dummy electrodes) 1-6-1 to 1-6-4 arranged in the electrode region 21 are not limited to the arrangement positions illustrated in FIG. 6. The number (four) of first electrodes (the temperature measurement dummy electrodes) 1-6 and the number of second electrodes (the reading electrodes) in the electrode region 21 are not limited to the number of first electrodes (the temperature measurement dummy electrodes) and the number of second electrodes (the reading electrodes) illustrated in FIG. 6.

As the heat conduction units used in the potential measurement device according to the sixth embodiment (Example 6 of a potential measurement device) according to the present technology, the heat conduction unit 3-1 used in the potential measurement device 1001 according to the first embodiment of the present technology, the heat conduction unit 3-2 used in the potential measurement device 1002 according to the second embodiment of the present technology, or the heat conduction unit 3-3 used in the potential measurement device 1003 according to the third embodiment of the present technology may be applied.

Incidentally, of the four first electrodes (the temperature measurement dummy electrodes) 1-6-1 to 1-6-4, at least one electrode may be substituted with a reading electrode 2-1 (an electrode also serving as a temperature measurement dummy electrode) used in the potential measurement device 1004 according to the fourth embodiment of the present technology.

A reference electrode used in the potential measurement device according to the sixth embodiment (Example 6 of a potential measurement device) of the present technology may be arranged in, for example, a region in the outer circumference of the electrode region 21.

The content described above in the potential measurement device according to the sixth embodiment (Example 6 of the potential measurement device) of the present technology can be applied to the potential measurement device according to the above-described first to fifth embodiments and potential measurement devices according to the seventh embodiment of the present technology to be described below as far as there is particularly no technical contradiction.

### 8. Seventh Embodiment (Example 7 of Potential Measurement Device)

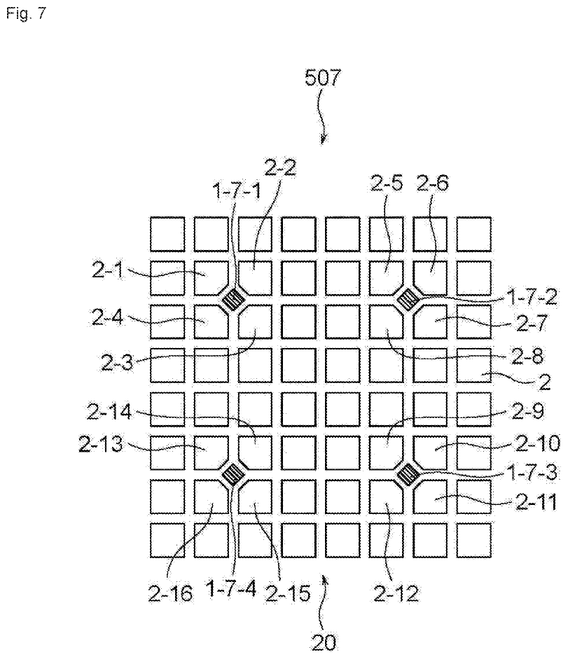

A potential measurement device according to a seventh embodiment (Example 7 of a potential measurement device) according to the present technology will be described with reference to FIG. 7.

FIG. 7 is a plan view illustrating an arrangement example of first electrodes (temperature measurement dummy electrodes) and second electrodes (reading electrodes) included in a potential measurement device according to the seventh embodiment to which the present technology is applied, and is a plan view of an arrangement configuration 507 of the first electrodes (the temperature measurement dummy electrodes) and the second electrodes (the reading electrodes) in detail.

In the arrangement configuration 507, a plurality of second electrodes (the reading electrodes) 2 are arranged in a 2-dimensional array form, and the electrode region 20 is formed.

In FIG. 7, a first electrode (the temperature measurement dummy electrode) 1-7-1 is arranged to be surrounded by four second electrodes (reading electrodes) 2-1 to 2-4. Specifically, the first electrode (the temperature measurement dummy electrode) 1-7-1 is arranged between the second electrodes (the reading electrodes) 2-1 and 2-2, is arranged between the second electrodes (the reading electrodes) 2-2 and 2-3, is arranged between the second electrodes (the reading electrodes) 2-3 and 2-4, and is arranged between the second electrodes (the reading electrodes) 2-4 and 2-1.

A first electrode (the temperature measurement dummy electrode) 1-7-2 is arranged to be surrounded by four second electrodes (reading electrodes) 2-5 to 2-8. Specifically, the first electrode (the temperature measurement dummy electrode) 1-7-2 is arranged between the second electrodes (the reading electrodes) 2-5 and 2-6, is arranged between the second electrodes (the reading electrodes) 2-6 and 2-7, is arranged between the second electrodes (the reading electrodes) 2-7 and 2-8, and is arranged between the second electrodes (the reading electrodes) 2-8 and 2-5.

A first electrode (the temperature measurement dummy electrode) 1-7-3 is arranged to be surrounded by four second electrodes (reading electrodes) 2-9 to 2-12. Specifically, the first electrode (the temperature measurement dummy electrode) 1-7-3 is arranged between the second electrodes (the reading electrodes) 2-9 and 2-10, is arranged between the second electrodes (the reading electrodes) 2-10 and 2-11, is arranged between the second electrodes (the reading electrodes) 2-11 and 2-12, and is arranged between the second electrodes (the reading electrodes) 2-12 and 2-9.

A first electrode (the temperature measurement dummy electrode) 1-7-4 is arranged to be surrounded by four second electrodes (reading electrodes) 2-13 to 2-16. Specifically, the first electrode (the temperature measurement dummy electrode) 1-7-4 is arranged between the second electrodes (the reading electrodes) 2-13 and 2-14, is arranged between the second electrodes (the reading electrodes) 2-14 and 2-15, is arranged between the second electrodes (the reading electrodes) 2-15 and 2-16, and is arranged between the second electrodes (the reading electrodes) 2-16 and 2-13.

The positions of the four first electrodes (the temperature measurement dummy electrodes) 1-7-1 to 1-7-4 disposed in the electrode region 20 are not limited to the positions of the first electrodes arranged in the electrode region illustrated in FIG. 7 as long as the positions of the first electrodes are disposed between the second electrodes (the reading electrodes). The number of first electrodes (the temperature measurement dummy electrodes) 1 is not limited to the number (four) of the first electrodes (the temperature measurement dummy electrodes) 1-7 illustrated in FIG. 6.

As the heat conduction units used in the potential measurement device according to the seventh embodiment (Example 7 of a potential measurement device) according to the present technology, the heat conduction unit 3-1 used in the potential measurement device 1001 according to the first embodiment of the present technology, the heat conduction unit 3-2 used in the potential measurement device 1002 according to the second embodiment of the present technology, or the heat conduction unit 3-3 used in the potential measurement device 1003 according to the third embodiment of the present technology may be applied.

A reference electrode used in the potential measurement device according to the seventh embodiment (Example 7 of a potential measurement device) of the present technology may be arranged in, for example, a region in the outer circumference of the electrode region 20.

The content described above in the potential measurement device according to the seventh embodiment (Example 7 of the potential measurement device) of the present technology can be applied to the potential measurement device according to the above-described first to sixth embodiments of the present technology as far as there is particularly no technical contradiction.

Embodiments of the present technology are not limited to the above-described embodiments and can be modified in various forms within the scope of the present technology without departing from the gist of the present technology.

The advantageous effects described in the present specification are merely exemplary and are not limited, and other advantageous effects may be achieved.

The present technology can be configured as follows.

[1]

A potential measurement device including:

a semiconductor substrate;
a wiring layer on the semiconductor substrate;
a first electrode on the wiring layer; and
a second electrode configured to detect an action potential of a cell on the wiring layer,
wherein a temperature measurement unit is formed on the semiconductor substrate, and
wherein a heat conduction unit and a plurality of wirings connected to the second electrode are formed in the wiring layer.

[2]

The potential measurement device according to [1], wherein the heat conduction unit and the first electrode are connected.

[3]

The potential measurement device according to [1], wherein the heat conduction unit is formed to extend to a region near the temperature measurement unit.

[4]

The potential measurement device according to [1],

wherein the heat conduction unit and the first electrode are connected, and
wherein the heat conduction unit is formed to extend from the first electrode to a region near the temperature measurement unit.

[5]

The potential measurement device according to any one of [1] to [4],

wherein the heat conduction unit includes first and second heat conduction members, and
wherein the first and second heat conduction members are connected.

[6]

The potential measurement device according to any one of [1] to [4],

wherein the heat conduction unit includes first and second heat conduction members,
wherein the first electrode and the first heat conduction member are connected, and
wherein the first and second heat conduction members are connected.

[7]

The potential measurement device according to any one of [1] to [4],

wherein the plurality of wirings are each connected through a via,
wherein the heat conduction unit includes first and second heat conduction members,
wherein the first and second heat conduction members are connected,
wherein the first heat conduction member and the via are formed in substantially the same layer, and
wherein the second heat conduction member and the wiring are formed in substantially the same layer.

[8]

The potential measurement device according to any one of [1] to [4],

wherein the plurality of wirings are each connected through a via,
wherein the heat conduction unit includes first and second heat conduction members,
wherein the first and second heat conduction members are connected,
wherein the first heat conduction member and the via are formed in substantially the same layer,
wherein the second heat conduction member and the wiring are formed in substantially the same layer,
wherein the first electrode and the first heat conduction member are connected, and
wherein the first and second heat conduction members are connected.

[9]

The potential measurement device according to any one of [1] to [4], wherein the heat conduction unit has a groove extending downward and a heat conduction material in which the groove is buried.

[10]

The potential measurement device according to any one of [1] to [4],

wherein the heat conduction unit has a groove extending downward and a heat conduction material in which the groove is buried, and wherein the heat conduction material and the first electrode are connected.

[11]

The potential measurement device according to any one of [1] to [10], further comprising:

an electrode region,
wherein, in the electrode region, a plurality of the second electrodes are arranged in a 2-dimensional array form, and
wherein at least one said first electrode is arranged on an outer circumference of the electrode region.

[12]

The potential measurement device according to any one of [1] to [10], further comprising:

an electrode region,
wherein, in the electrode region, a plurality of the second electrodes are arranged in a 2-dimensional array form,
wherein at least one said first electrode is arranged on an outer circumference of the electrode region, and
wherein the heat conduction unit and said at least one first electrode are connected.

[13]

The potential measurement device according to any one of [1] to [10], further comprising:

an electrode region,
wherein, in the electrode region, at least one said first electrode and a plurality of the second electrodes are arranged in a 2-dimensional array form.

[14]

The potential measurement device according to any one of [1] to [10], further comprising:

an electrode region,
wherein, in the electrode region, at least one said first electrode and a plurality of the second electrodes are arranged in a 2-dimensional array form, and
wherein the heat conduction unit and said at least one first electrode are connected.

[15]

The potential measurement device according to any one of [1] to [14],

wherein at least two second electrode are included, and
wherein at least one said first electrode is arranged between said at least two electrodes.

[16]

The potential measurement device according to any one of [1] to [14],

wherein at least two second electrode are included, and
wherein at least one said first electrode is arranged between said at least two electrodes, and
wherein the heat conduction unit and said at least one first electrode are connected.

[17]

A potential measurement device including:

a semiconductor substrate;
a wiring layer on the semiconductor substrate; and
a third electrode configured to detect an action potential of a cell on the wiring layer,
wherein a temperature measurement unit is formed on the semiconductor substrate, and
wherein a plurality of wirings connected to a heat conduction unit and the third electrode are formed in the wiring layer.

[18]

The potential measurement device according to [17], wherein the heat conduction unit and the third electrode are connected.

[19]

The potential measurement device according to [17] or [18], wherein the heat conduction unit is formed to extend to a region near the temperature measurement unit.

[20]

The potential measurement device according to [17],

wherein the heat conduction unit and the third electrode are connected, and
wherein the heat conduction unit is formed to extend from the third electrode to a region near the temperature measurement unit.

[21]

The potential measurement device according to any one of [17] to [20],

wherein the heat conduction unit includes first and second heat conduction members, and
wherein the first and second heat conduction members are connected.

[22]

The potential measurement device according to any one of [17] to [20],

wherein the heat conduction unit includes first and second heat conduction members,
wherein the third electrode and the first heat conduction member are connected, and
wherein the first and second heat conduction members are connected.

[23]

The potential measurement device according to any one of [17] to [20],

wherein the plurality of wirings are each connected through a via,
wherein the heat conduction unit includes first and second heat conduction members,
wherein the first and second heat conduction members are connected,
wherein the first heat conduction member and the via are formed in substantially the same layer, and
wherein the second heat conduction member and the wiring are formed in substantially the same layer.

[24]

The potential measurement device according to any one of [17] to [20],

wherein the plurality of wirings are each connected through a via,
wherein the heat conduction unit includes first and second heat conduction members,
wherein the first and second heat conduction members are connected,
wherein the first heat conduction member and the via are formed in substantially the same layer,
wherein the second heat conduction member and the wiring are formed in substantially the same layer,
wherein the first electrode and the first heat conduction member are connected, and
wherein the first and second heat conduction members are connected.

[25]

The potential measurement device according to any one of [17] to [20], wherein the heat conduction unit has a groove extending downward and a heat conduction material in which the groove is buried.

[26]

The potential measurement device according to any one of [17] to [20],

wherein the heat conduction unit has a groove extending downward and a heat conduction material in which the groove is buried, and
wherein the heat conduction material and the first electrode are connected.

[27]

The potential measurement device according to any one of [17] to [26], further comprising:

an electrode region,
wherein, in the electrode region, a plurality of the second electrodes are arranged in a 2-dimensional array form, and
wherein at least said one first electrode is arranged on an outer circumference of the electrode region.

[28]

The potential measurement device according to any one of [17] to [26], further comprising:

an electrode region,
wherein, in the electrode region, a plurality of the second electrodes are arranged in a 2-dimensional array form,
wherein at least one said first electrode is arranged on an outer circumference of the electrode region, and
wherein the heat conduction unit and said at least one first electrode are connected.

[29]

The potential measurement device according to any one of [17] to [26], further comprising:

an electrode region,
wherein, in the electrode region, at least one said first electrode and a plurality of the second electrodes are arranged in a 2-dimensional array form.

[30]

The potential measurement device according to any one of [17] to [26], further comprising:

an electrode region,
wherein, in the electrode region, at least one said first electrode and a plurality of the second electrodes are arranged in a 2-dimensional array form, and
wherein the heat conduction unit and said at least one first electrode are connected.

[31]

The potential measurement device according to any one of [17] to [30],

wherein said at least two second electrode are included, and
wherein at least one said first electrode is arranged between said at least two electrodes.

[32]

The potential measurement device according to any one of [17] to [30],

wherein at least two second electrode are included, and
wherein at least one said first electrode is arranged between said at least two electrodes, and
wherein the heat conduction unit and said at least one first electrode are connected.

### REFERENCE SIGNS LIST

1 (1-5-1, 1-5-2, 1-5-3, 1-5-4, 1-6-1, 1-6-2, 1-6-3, 1-6-4, 1-7-1, 1-7-2, 1-7-3, 1-7-4) First electrode (temperature measurement dummy electrode)
2 (2-2, 2-3, 2-4, 2-5, 2-6, 2-7, 2-8, 2-9, 2-10, 2-11,2-12, 2-13, 2-14, 2-15, 2-16) Second electrode (reading electrode)
2-1 Third electrode
3 (3-1, 3-2, 3-3, 3-4) Heat conduction unit
4 (4-1, 4-2, 4-3, 4-4), 40 (40-1, 40-2,) First heat conduction member
5 (5-1, 5-2, 5-3, 5-4), 50 (50-1, 50-2, 50-3, 50-4) Second heat conduction member
6 Temperature measurement unit
7 Semiconductor substrate
8 Amplifier
11 (11-1, 11-2, 11-3, 11-4) Wiring
12 (12-1, 12-2, 12-3, 12-4, 12-5) Via
13 Insulation film (inter-layer insulation film)
80 Cell
90 Culture solution
505, 506, 507 Arrangement configuration of first electrode (temperature measurement dummy electrode) and second electrode (reading electrode)
1001, 1002, 1003, 1004 Potential measurement device
H Heat conduction path

1. A potential measurement device comprising:
a semiconductor substrate;
a wiring layer on the semiconductor substrate;
a first electrode on the wiring layer; and
a second electrode configured to detect an action potential of a cell on the wiring layer,
wherein a temperature measurement unit is formed on the semiconductor substrate, and
wherein a heat conduction unit and a plurality of wirings connected to the second electrode are formed in the wiring layer.

2. The potential measurement device according to claim 1, wherein the heat conduction unit and the first electrode are connected.

3. The potential measurement device according to claim 1, wherein the heat conduction unit is formed to extend to a region near the temperature measurement unit.

4. The potential measurement device according to claim 1,
wherein the heat conduction unit and the first electrode are connected, and
wherein the heat conduction unit is formed to extend from the first electrode to a region near the temperature measurement unit.

5. The potential measurement device according to claim 1,
wherein the heat conduction unit includes first and second heat conduction members, and
wherein the first and second heat conduction members are connected.

6. The potential measurement device according to claim 1,
wherein the heat conduction unit includes first and second heat conduction members,
wherein the first electrode and the first heat conduction member are connected, and
wherein the first and second heat conduction members are connected.

7. The potential measurement device according to claim 1,
wherein the plurality of wirings are each connected through a via,
wherein the heat conduction unit includes first and second heat conduction members,
wherein the first and second heat conduction members are connected,
wherein the first heat conduction member and the via are formed in substantially the same layer, and
wherein the second heat conduction member and the wiring are formed in substantially the same layer.

8. The potential measurement device according to claim 1,
wherein the plurality of wirings are each connected through a via,
wherein the heat conduction unit includes first and second heat conduction members,
wherein the first and second heat conduction members are connected,
wherein the first heat conduction member and the via are formed in substantially the same layer,
wherein the second heat conduction member and the wiring are formed in substantially the same layer,
wherein the first electrode and the first heat conduction member are connected, and
wherein the first and second heat conduction members are connected.

9. The potential measurement device according to claim 1, wherein the heat conduction unit has a groove extending downward and a heat conduction material in which the groove is buried.

10. The potential measurement device according to claim 1,
wherein the heat conduction unit has a groove extending downward and a heat conduction material in which the groove is buried, and
wherein the heat conduction material and the first electrode are connected.

11. The potential measurement device according to claim 1, further comprising:
an electrode region,
wherein, in the electrode region, a plurality of the second electrodes are arranged in a 2-dimensional array form, and
wherein at least one said first electrode is arranged on an outer circumference of the electrode region.

12. The potential measurement device according to claim 1, further comprising:
an electrode region,
wherein, in the electrode region, a plurality of the second electrodes are arranged in a 2-dimensional array form,
wherein at least one said first electrode is arranged on an outer circumference of the electrode region, and
wherein the heat conduction unit and said at least one first electrode are connected.

13. The potential measurement device according to claim 1, further comprising:
an electrode region,
wherein, in the electrode region, at least one said first electrode and a plurality of the second electrodes are arranged in a 2-dimensional array form.

14. The potential measurement device according to claim 1, further comprising:
an electrode region,
wherein, in the electrode region, at least one first electrode and a plurality of the second electrodes are arranged in a 2-dimensional array form, and
wherein the heat conduction unit and at least one first electrode are connected.

15. The potential measurement device according to claim 1,
wherein at least two second electrode are included, and
wherein the at least one first electrode is arranged between said at least two electrodes.

16. The potential measurement device according to claim 1,
wherein at least two second electrode are included, and
wherein at least one first electrode is arranged between said at least two electrodes, and
wherein the heat conduction unit and said at least one first electrode are connected.

17. A potential measurement device comprising:
a semiconductor substrate;
a wiring layer on the semiconductor substrate; and
a third electrode configured to detect an action potential of a cell on the wiring layer,
wherein a temperature measurement unit is formed on the semiconductor substrate, and
wherein a plurality of wirings connected to a heat conduction unit and the third electrode are formed in the wiring layer.

18. The potential measurement device according to claim 17, wherein the heat conduction unit and the third electrode are connected.

19. The potential measurement device according to claim 17, wherein the heat conduction unit is formed to extend to a region near the temperature measurement unit.

20. The potential measurement device according to claim 17,
wherein the heat conduction unit and the third electrode are connected, and
wherein the heat conduction unit is formed to extend from the third electrode to a region near the temperature measurement unit.
